(19) United States
(12) Patent Application Publication
(10) Pub. No.: US 2024/0407978 A1
(43) Pub. Date: Dec. 12, 2024

(54) PIGMENT SUSPENSION AND COSMETIC AGENT PREPARED USING THE PIGMENT SUSPENSION

(71) Applicant: HENKEL AG & CO. KGAA, Düsseldorf (DE)

(72) Inventors: Gabriele WESER, Essen (DE); Caroline KRIENER, Düsseldorf (DE)

(21) Appl. No.: 18/697,593

(22) PCT Filed: Sep. 5, 2022
(86) PCT No.: PCT/EP2022/074626

§ 371 Date: Apr. 1, 2024

(30) Foreign Application Priority Data
Oct. 4, 2021 (DE) 10 2021 211 138.6

(51) Int. Cl. A61K 8/04; A61K 8/26; A61K 8/86; A61Q 5/06

(52) U.S. Cl. CPC A61K 8/044; A61K 8/26; A61K 8/86; A61Q 5/06

(57) ABSTRACT

A pigment suspension including at least one aluminum pigment and at least one polyethylene glycol having an average molecular weight of from 200 g/mol to 35,000 g/mol is described. A cosmetic agent including the pigment suspension and a cleansing and/or nurturing ingredient is also described.

The subject matter of the present invention is a pigment suspension comprising a pigment and a carrier medium. A further subject matter is a cosmetic agent which contains the pigment suspension and a cleansing and/or nurturing ingredient.

Pigments are frequently used in lacquers, paints, printing inks, powder coatings, cosmetics, or plastics for coloring. Colors, lacquers, printing inks, cosmetics, and powder coatings are liquid or powdery coating materials which are applied to surfaces in order to obtain both improved or altered optical and also physical properties.

Changing the shape and color of keratinous fibers, in particular hair, represents an important area of modern cosmetics. To change the hair color, the skilled artisan is familiar with a variety of dyeing systems depending on the dyeing requirements. Oxidation dyes are typically used for permanent, intense dyeing with good fastness properties and good gray coverage. Such dyes typically contain oxidation dye precursors, known as developer components, and coupler components, which together form the actual dyes under the influence of oxidizing agents, for example hydrogen peroxide. Oxidation dyes are characterized by very long-lasting color results.

When using direct dyes, dyes which are already formed diffuse out of the dyeing agent into the hair fiber. In comparison with oxidative hair dyeing, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

Ash blond is a currently fashionable hair color. This color tone is a cool, matte blond with a light gray tone. The color is well suited to apply accents with highlights or balayage. Such effects primarily have a good effect in long hair.

The perfect starting color for ash blond is blond. With the aid of a silver shampoo, blond hair can be converted into a cool ash blond by the blue and/or violet pigments and/or direct dyes contained therein.

In the case of blue direct dyes, the problem often arises that they only adhere to the hair unevenly and thus lead to a correspondingly non-uniform color result.

It is an object of the present invention to provide a cosmetic composition which is capable of matting keratinous fibers, in particular blond keratinous fibers.

It has been shown that a pigment suspension containing at least one aluminum pigment uniformly mats blond hair and gives it a light gray tone. Dark hair can be given a silky shimmer by applying the pigment suspension.

Accordingly, a first subject matter of the application is a pigment suspension comprising a) at least one aluminum pigment and b) at least one polyethylene glycol having an average molecular weight of 200 to 35,000 g/mol.

As a first ingredient essential to the invention, the pigment suspensions contain at least one aluminum pigment.

Aluminum pigments are used in lacquers, inks and plastics, but also in decorative cosmetics, for example in nail polishes.

The at least one aluminum pigment preferably has a small substrate plate.

Small substrate plates made of aluminum can be produced, inter alia, by punching them out of aluminum foil or by customary milling and spraying techniques. For example, small aluminum plates are obtained using the Hall process, a wet milling method.

A small substrate plate made of aluminum preferably has an average thickness of at most 150 nm, preferably less than 50 nm, more preferably less than 30 nm, particularly preferably at most 25 nm, for example at most 20 nm. The average thickness of the small substrate plates is at least 1 nm, preferably at least 2.5 nm, particularly preferably at least 5 nm, for example at least 10 nm. Preferred ranges for the thickness of the small substrate plate are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm and 10 to 20 nm. Preferably, each substrate plate has as uniform a thickness as possible.

The size of the small substrate plate can be tailored to the respective application purpose, for example to the desired effect on a keratinous material. In general, the small substrate plates made of aluminum have an average maximum diameter of approximately 2 to 200 μm, in particular approximately 5 to 100 μm. As a rule, the small substrate plates made of a mica have a mean maximum diameter of approximately 1 to 200 μm, in particular approximately 5 to 100 μm and even more preferably approximately 5 to 25 μm.

In a preferred embodiment, the form factor (aspect ratio), expressed by the ratio of the average size to the average thickness, is at least 80, preferably at least 200, more preferably at least 500, particularly preferably more than 750. In this case, the average size of the uncoated small substrate plates is understood to mean the d50 value of the uncoated small substrate plates. Unless stated otherwise, the d50 value was determined using a device of the Sympatec Helos type, having QUIXEL wet dispersion. To prepare the sample, the sample to be investigated was pre-dispersed in isopropanol for a period of 3 minutes.

The small substrate plates can have different shapes. Small substrate plates can be, for example, lamellar or lenticular metal plates or else so-called vacuum metalized pigments (VMP). Lamellar small substrate plates are characterized by an irregularly structured edge, and are also referred to as “cornflakes” due to their appearance. Lenticular small substrate plates have a substantially regular round edge and are also referred to as “silver dollars” due to their appearance.

The small substrate plates made of aluminum can be passivated, for example by anodizing (oxide layer) or chromatizing.

By means of a coating, the surface properties and/or optical properties of the pigment can be altered and the mechanical and chemical resistance of the pigments can be increased. For example, only the upper and/or lower side of the small substrate plate can be coated, the side faces being omitted. Preferably, the entire surface of the optionally passivated small substrate plates, including the side surfaces, is covered by the layer. The small substrate plates are preferably completely encased by the coating.

The coating can consist of one or more layers. In a preferred embodiment, the coating has only one layer A. In a likewise preferred embodiment, the coating has a total of at least two, preferably two or three, layers. It may be preferred for the coating to have two layers A and B, wherein layer B is different from layer A. Layer A is preferably located between layer B and the surface of the small substrate plate. In yet another preferred embodiment, the coating has three layers A, B and C. In this embodiment, the layer A is located between the layer B and the surface of the small substrate plate, and a layer C, which is different from the underlying layer B, is located on layer B.

Suitable materials for the layers A and, in some cases, B and C are all substances which can be applied permanently to the small substrate plates. The materials should preferably be applied in a film-like manner. Preferably, the entire surface of the, in some cases, passivated small substrate plates, including the side surfaces, is encased by layer A or by layers A and B or by layers A, B and C.

The layers can in particular each contain at least one metal oxide (hydrate).

It is preferred that the metal oxide (hydrate) is selected from the group consisting of silicon (di) oxide, silicon oxide hydrate, aluminum oxide, aluminum oxide hydrate, boron oxide, germanium oxide, manganese oxide, magnesium oxide, iron oxide, cobalt oxide, chromium oxide, titanium dioxide, vanadium oxide, zirconium oxide, tin oxide, zinc oxide, and mixtures thereof.

In the case of pigments having a small substrate plate made of metal or a metal alloy, layer A preferably has at least one low-refractive-index metal oxide and/or metal oxide hydrate. Low-refractive materials have a refractive index of at most 1.8, preferably at most 1.6.

The low-refractive metal oxide (hydrate) s suitable for the layer A include, for example, silicon (di) oxide, silicon oxide hydrate, aluminum oxide, aluminum oxide hydrate, boric oxide, germanium oxide, manganese oxide, magnesium oxide, and mixtures thereof, with silicon dioxide being preferred. The layer A preferably has a thickness from 1 to 100 nm, particularly preferably 5 to 50 nm, in particular preferably 5 to 20 nm.

Layer B, if present, is different from layer A and, in the case of pigments having a small substrate plate made of metal or a metal alloy, may contain at least one high-refractive metal oxide (hydrate). Highly refractive materials have a refractive index of at least 1.9, preferably at least 2.0, and particularly preferably at least 2.4. The layer B preferably comprises at least 95 wt. %, particularly preferably at least 99 wt. %, of highly refractive metal oxide(s).

If layer B contains a (high refractive index) metal oxide, it preferably has a thickness of at least 50 nm. The thickness of layer B is preferably no more than 400 nm, particularly preferably at most 300 nm.

Highly refractive metal oxides suitable for layer B are, for example, selectively light-absorbing (i.e., colored) metal oxides such as iron (III) oxide (α- and γ-Fe2O3, red), cobalt (II) oxide (blue), chromium (III) oxide (green), titanium (III) oxide (blue, usually in a mixture with titanium oxynitrides and titanium nitrides) and vanadium (V) oxide (orange) and mixtures thereof. Also suitable are colorless, highly-refractive oxides such as titanium dioxide and/or zirconium oxide.

According to a preferred embodiment, the pigments with a small substrate plate made of aluminum have a further layer C comprising a metal oxide (hydrate) which is different from the underlying layer B. Suitable metal oxides are, for example, silicon (di) oxide, silicon oxide hydrate, aluminum oxide, aluminum oxide hydrate, zinc oxide, tin oxide, titanium dioxide, zirconium oxide, iron (III) oxide and chromium (III) oxide. Silicon dioxide is preferred.

In the case of pigments having a small substrate plate made of metal or a metal alloy, the layer C preferably has a thickness of 10 to 500 nm, particularly preferably 50 to 300 nm.

Suitable pigments based on a small substrate plate made of a metal or a metal alloy are, for example, the pigments Alegrace® Marvelous, Alegrace® Gorgeous, or Alegrace® Aurous from Schlenk Metallic pigments GmbH.

The amount of aluminum pigment in the pigment suspension depends in particular on the nature of the pigment/pigments and the application thereof. Preferably, the amount of pigment is between 0.05 and 5 wt. %, more preferably between 0.1 and 4 wt. % and very particularly preferably between 0.2 and 3 wt. %, based on the total weight of the pigment suspension.

As a second ingredient essential to the invention, the pigment suspension comprises at least one polyethylene glycol having an average molecular weight of 200 to 35,000 g/mol.

Polyethylene glycol (PEG) is, depending on the chain length, a liquid, pasty or solid polymer with the general molecular formula C₂ₙH₄ₙ₊₂Oₙ₊₁.

Polyethylene glycols having an average molecular weight of 200 to 400 g/mol are liquid, polyethylene glycols having an average molecular weight of >400 to 600 g/mol are pasty, and polyethylene glycols having an average molecular weight of ≥1000 g/mol are solid.

It has been found that the presence of at least two different polyethylene glycols in the pigment suspension improves the ability to distribute the pigment suspension on the keratinous fibers, thereby resulting in particularly homogeneous matting.

Accordingly, it is preferable for the pigment suspension to comprise at least two polyethylene glycols having an average molecular weight of 200 to 35,000 g/mol. It is even more preferable if the pigment suspension contains at least one liquid or pasty polyethylene glycol and at least one solid polyethylene glycol.

In a particularly preferred embodiment, the pigment suspension comprises at least one first polyethylene glycol having an average molecular weight of 200 to 600 g/mol and at least one second polyethylene glycol having an average molecular weight of 1000 to 35,000 g/mol.

In a very particularly preferred embodiment, the pigment suspension contains as a first polyethylene glycol having an average molecular weight of 200 to 600 g/mol a polyethylene glycol having an average molecular weight of 400 g/mol.

In a further very particularly preferred embodiment, the pigment suspension contains as a second polyethylene glycol having an average molecular weight of 1,000 to 35000 g/mol a polyethylene glycol having an average molecular weight of 6,000 g/mol.

In a particularly preferred embodiment, the proportion of the first polyethylene glycol in the pigment suspension is higher than the proportion of the second polyethylene glycol.

In a preferred embodiment, the pigment suspension is characterized in that the first polyethylene glycol having an average molecular weight of from 200 to 600 g/mol and the second polyethylene glycol having an average molecular weight of from 1000 to 35,000 g/mol are present in a weight ratio in the range from 15:1 to 1:1, preferably 12:1 to 2:1 and in particular 10:1 to 5:1. In a very preferred embodiment, the method is characterized in that the weight ratio of the first polyethylene glycol and the second polyethylene glycol in the agent is in the range from 7:1 to 9:1, most preferably 8:1.

Particularly good results were obtained when the pigment suspension contained—based on the total weight of the pigment suspension—the at least on polyethylene glycol having an average molecular weight of 200 to 35,000 g/mol in a total amount from 95 to 99.95 wt. %, preferably from 96 to 99.9 wt. % and very particularly preferably from 97 to 99.8 wt. %.

The pigment suspensions can contain small amounts, preferably up to 4.9 wt. % and more preferably up to 2 wt. %, based in each case on the total weight of the pigment suspension, of further ingredients which can comprise in particular further solvents, for example water, isopropyl alcohol and/or ethyl acetate, bases and/or corrosion inhibitors.

The pigment suspension can, as it is applied, be used to mat keratinous fibers, in particular blond keratinous fibers.

Alternatively, the pigment suspension can be combined with at least one essential ingredient of a cosmetic agent for hair treatment, in particular a shampoo, a rinse or a hair treatment.

A second subject matter of the present invention relates to a cosmetic agent. The cosmetic agent comprises i) a pigment suspension according to the invention and ii) a cleansing and/or nurturing ingredient.

The cosmetic agent is prepared, for example, by combining a pigment suspension according to the present invention with at least one cleansing surfactant and/or at least one nurturing ingredient selected from the group of quaternary compounds, silicone oils, protein hydrolyzates, vegetable oils, vitamins, vitamin precursors, plant extracts, monosaccharides, oligosaccharides, amino acids, purine alkaloids, glycols, and mixtures. In this way, cosmetic agents can be provided which, in addition to the desired matting, give the keratinous fibers treated therewith a further cosmetic advantage (cleansing and/or nurturing).

Cosmetic agents of this kind can be used, for example, in the cleansing and/or nurturing of keratinous fibers, in particular human hair.

A cleansing and matting cosmetic agent is characterized in that it contains at least one cleansing surfactant. In the context of this application, cleansing surfactants are understood to mean anionic surfactants, amphoteric/zwitterionic surfactants and non-ionic surfactants.

It is preferred that the cleansing and matting cosmetic agent comprises a surfactant mixture of anionic and amphoteric/zwitterionic surfactants.

Suitable cleansing surfactants are used in the cosmetic agent—based on the total weight thereof—preferably in amounts of 0.1 to 30 wt. %, more preferably of 1 to 20 wt. %, particularly preferably of 2 to 17.5 wt. %, and in particular of 3 to 15 wt. %.

The amount of pigment suspension in the cleansing and matting cosmetic agent—based on the total weight thereof—is from 10 to 99 wt. %, preferably from 40 to 99 wt. %.

In addition to the cleansing surfactant and the pigment suspension according to the invention, the cleansing and matting cosmetic agent may contain water and/or a mixture of water and at least one C1-C4 alcohol.

The suitable anionic surfactants which can be used in the cosmetic agent include:
straight-chain or branched, saturated or mono- or polyunsaturated alkylsulfonates having 8 to 24 C atoms,
linear alpha-olefin sulfonates having 8 to 24 C atoms,
alkyl sulfates and alkyl polyglycol ether sulfates of the formula R—O—(CH₂—CH₂O)ₙ—SO₃X, where R preferably represents a straight-chain or branched, saturated or mono- or polyunsaturated alkyl or alkenyl radical having 8 to 24 carbon atoms, n represents 0 or 1 to 12 and X represents an alkali metal or alkaline earth metal ion or protonated triethanolamine or the ammonium ion, sulfonates of unsaturated fatty acids having 8 to 24 C atoms and 1 to 6 double bonds.

Particularly preferred anionic surfactants are straight chain or branched alkyl ether sulfates which contain an alkyl radical having 8 to 18 and in particular 10 to 16 C atoms and 1 to 6 and in particular 2 to 4 ethylene oxide units. Very particularly preferably, the surfactant mixture of anionic and amphoteric/zwitterionic surfactants contains sodium lauryl ether sulfate (INCI: sodium laureth sulfate) and very particularly preferably sodium lauryl ether sulfate having 2 ethylene oxide units.

Further particularly preferred anionic surfactants are straight-chain or branched alkyl sulfonates which contain an alkyl radical having 8 to 18 and in particular 10 to 16 C atoms.

Particularly preferable are the sodium, magnesium and/or triethanolamine salts of linear or branched lauryl, tridecyl and/or myristyl sulfates having a degree of ethoxylation from 2 to 4.

Suitable amphoteric/zwitterionic surfactants can be added in the cleansing and matting cosmetic agent—based on the total weight thereof—preferably in amounts of 0.1 to 25 wt. %, more preferably 0.25 to 20 wt. %, particularly preferably 0.5 to 15.0 and in particular from 1 to 10 wt. %.

Suitable amphoteric/zwitterionic surfactants can be selected from compounds of the following formulae (i) to (v), in which the radical R is in each case a straight-chain or branched, saturated or mono- or polyunsaturated alkyl or alkenyl radical having 8 to 24 carbon atoms,

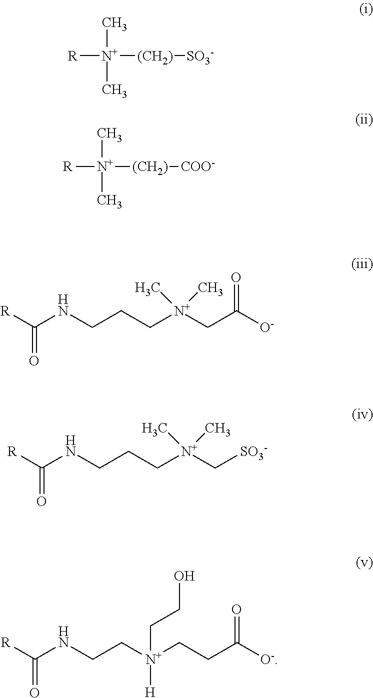

The particularly suitable amphoteric/zwitterionic surfactants include the surfactants known under the INCI designation “cocamidopropyl betaine” and “disodium cocoamphodiacetate.”

The amount of pigment suspension in the cleansing and matting cosmetic agent depends on the desired matting effect and—based on the total weight thereof—can be from 10 to 99 wt. %.

Alternatively, the cosmetic agent can be characterized in that it comprises at least one nurturing ingredient selected from the group of quaternary compounds, silicone oils, protein hydrolyzates, vegetable oils, vitamins, vitamin precursors, plant extracts, monosaccharides, oligosaccharides, amino acids, purine alkaloids, glycols, and mixtures thereof.

Suitable protein hydrolyzates include, for example, protein hydrolyzates from silk, pashmina, cashmere wool, merino wool, mohair, wheat, corn, rice, potatoes, soybeans, almonds, peas, algae, collagen and/or milk.

Suitable vegetable oils include, for example: amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, coconut oil, linseed oil, macadamia nut oil, corn oil, almond oil, marula oil, evening primrose oil, olive oil, palm oil, palm kernel oil, para nut oil, pecan nut oil, peach kernel oil, rapeseed oil, castor oil, sea buckthorn pulp oil, sea buckthorn seed oil, sesame oil, soy oil, sunflower oil, grapeseed oil, walnut oil, wild rose oil and/or wheat germ oil.

Suitable vitamins include vitamin A, vitamin E, vitamin B2, vitamin B5, vitamin B7 and/or folic acid

Vitamin precursors may comprise, for example, panthenol.

Suitable plant extracts include, for example, extracts of tea tree oil, lavender, nettle, rosemary, chamomile, marigold, aloe vera, ginseng, soybeans, birch and/or wheat.

Suitable monosaccharides are, for example, erythrose, threose, ribose, arabinose, xylose, lyxose, allose, altrose, glucose, mannose, gulose, idose, galactose and/or fructose.

For example, lactose, sucrose, maltose and/or raffinose can be used as oligosaccharides.

The group of amino acids comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and/or valine.

Suitable purine alkaloids include, for example, caffeine, theophylline and/or theobromine.

Suitable glycols can be selected from ethylene glycol, 1,2-propylene glycol, 1,3-propylene glycol, 2-methyl-1,3-propandiol, 1,2-butylene glycol, 1,3-butylene glycol, 1,4-butylene glycol, 1,2-pentanediol, 1,5-pentanediol, 1,2-hexanediol, 1,6-hexanediol, 1,2-octanediol, 1,8-octanediol, cis-1,4-dimethylolcyclohexane, trans-1,4-dimethylolcyclohexane, any isomeric mixtures of cis- and trans-1,4-dimethylolcyclohexane, diethylene glycol, dipropylene glycol, PPG-10 butanediol (INCI), and mixtures thereof. If present, the glycols are used in addition to the polyethylene glycols.

A preferred cosmetic agent for matting and nurturing of keratinous fibers is characterized in that it contains at least one quaternary compound.

Quaternary compounds are monomeric cationic or amphoteric ammonium compounds, monomeric amines, aminoamides, polymeric cationic ammonium compounds and polymeric amphoteric ammonium compounds. The amount of the at least one quaternary compound in the matting and nourishing cosmetic agent is—based on the total weight thereof—from 0.1 to 10 wt. %. This amount is also not fallen below or exceeded when a mixture of different compounds of the quaternary compounds is used.

A very preferable cosmetic agent for matting and nurture of keratinous fibers is characterized in that it contains at least one quaternary compound selected from at least one of the following groups:
i. the monoalkyl quats and/or
ii. the esterquats and/or
iii. the quaternary imidazolines of the formula (Tkat4),

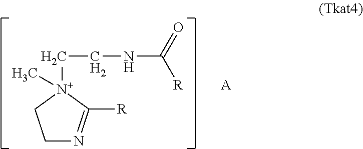

in which the functional groups R independently of one another each represent a saturated or unsaturated, linear or branched hydrocarbon functional group having a chain length of 8 to 30 carbon atoms, and A represents a physiologically acceptable anion, and/or
iv. the amines and/or cationized amines, and/or
v. poly(methacryloyloxyethyltrimethylammonium) compounds and/or;
vi. quaternized cellulose derivatives, in particular polyquaternium-10, polyquaternium-24, polyquaternium-27, polyquaternium-67, polyquaternium-72, and/or
vii. cationic alkyl polyglycosides, and/or
viii. cationized honey, and/or
ix. cationic guar derivatives, and/or
x. chitosan, and/or
xi. polymeric dimethyldiallylammonium salts and copolymers thereof with esters and amides of acrylic acid and methacrylic acid, in particular polyquaternium-7, and/or
xii. copolymers of vinylpyrrolidone with quaternized derivatives of dialkylaminoalkyl acrylate and dialkylaminoalkyl methacrylate, in particular polyquaternium-11, and/or
xiii. vinylpyrrolidone-vinylimidazolium methochloride copolymers, in particular polyquaternium-16 and/or
xiv. quaternized polyvinyl alcohol and/or
XV. polyquaternium-74,
and mixtures thereof are contained therein.

Monoalkyl quats are cationic ammonium compounds of the formula (Tkat1) form the first group of suitable quaternary compounds.

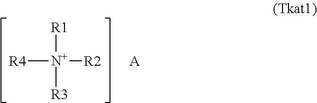

In the formula (Tkat1), R1, R2, R3 and R4 independently of one another each represent hydrogen, a methyl group, a phenyl group, a benzyl group, a saturated, branched or unbranched alkyl radical having a chain length of 8 to 30 carbon atoms, which may optionally be substituted with one or more hydroxyl groups. A represents a physiologically acceptable anion, for example halides such as chloride or bromide as well as methosulfates.

Examples for compounds of the formula (Tkat1) are lauryl trimethyl ammonium chloride, cetyl trimethyl ammonium chloride, cetyl trimethyl ammonium bromide, cetyl trimethyl ammonium methosulfate, dicetyl dimethyl ammonium chloride, tricetyl methyl ammonium chloride, stearyl trimethyl ammonium chloride, distearyl dimethyl ammonium chloride, lauryl dimethyl benzyl ammonium chloride, behenyl trimethyl ammonium chloride, behenyl trimethyl ammonium bromide, behenyl trimethyl ammonium methosulfate.

Esterquats are quaternary compounds according to the formula (Tkat2) and form a further preferred group of quaternary compounds.

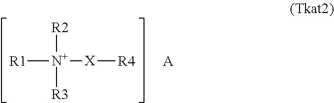

Herein, the radicals R1, R2, and R3 are each independent of one another and may be the same or different. The radicals R1, R2, and R3 denote:
a branched or unbranched alkyl radical having 1 to 4 carbon atoms which can contain at least one hydroxyl group, or
a saturated or unsaturated, branched or unbranched or a cyclically saturated or unsaturated alkyl radical having 6 to 30 carbon atoms which can contain at least one hydroxyl group, or
an aryl or alkaryl radical, for example, phenyl or benzene,
the radical (—X—R4), with the proviso that at most 2 of the radicals R1, R2 or R3 may represent this radical.

The radical-(X—R4) is present at least 1 to 3 times.

Herein, X represents:
1) —(CH2)n— with n=1 to 20, preferably n=1 to 10, and particularly preferably n=1 to 5, or
2) —(CH2-CHR5-O)n-, where n=1 to 200, preferably 1 to 100, particularly preferably 1 to 50 and particularly preferably 1 to 20 with R5 denoting hydrogen, methyl or ethyl,
3) a hydroxyalkyl group having one to four carbon atoms, which may be branched or unbranched, and which contains at least one and at most 3 hydroxyl groups. Examples are: —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl radicals,
and R4 represents:
1) R6-O—CO—, where R6 is a saturated or unsaturated, branched or unbranched or a cyclic saturated or unsaturated alkyl radical having 6 to 30 carbon atoms, which can contain at least one hydroxy group, and which optionally can be oxalkylated furthermore with 1 to 100 ethylene oxide units and/or 1 to 100 propylene oxide units, or
2) R₇—O—CO—, where R7 is a saturated or unsaturated, branched or unbranched or a cyclic saturated or unsaturated alkyl radical having 6 to 30 carbon atoms, which can contain at least one hydroxy group, and which optionally can be oxalkylated furthermore with 1 to 100 ethylene oxide units and/or 1 to 100 propylene oxide units, and
and A represents a physiologically acceptable organic or inorganic anion and is defined at this point as representative of all the structures described below. The anion of all the described cationic compounds is selected from the halide ions, fluoride, chloride, bromide, iodide, sulfates of general formula RSO₃⁻, in which R has the meaning of saturated or unsaturated alkyl radicals having 1 to 4 carbon atoms, or anionic radicals of organic acids such as maleate, fumarate, oxalate, tartrate, citrate, lactate or acetate.

Such products are marketed, for example, under the names Rewoquat®, Stepantex®, Dehyquart®, Armocare® and Quartamin®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Quartamin® BTC 131 are examples of these esterquats.

Further preferred quaternary compounds are cationic betaine esters of formula (Tkat3).

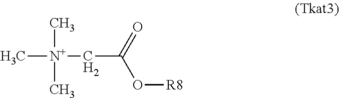

R8 corresponds in its meaning to R7.

Further suitable quaternary compounds have the general formula (I).

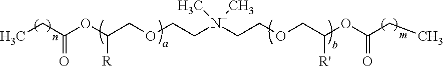

where
n and m independently of one another represent integers between 5 and 40, with the
proviso that n+m≥38; particularly preferably n=m; most preferably n=m=20.
a and b independently of one another represent integers between 1 and 10; particularly independently of one another 1, 2, 3, 4 or 5, preferably the equation a+2≥b≥a−2 and is most preferably a=b=3.
R and R′ are independently selected from —H and —CH₃; preferably, R=R′ applies, so that preferably either PEG or PPG diester quats are used; most preferably, R=R′=—CH₃ applies
X— a physiologically acceptable anion, a halide such as chloride, bromide or iodide, toluenesulfonate, methosulfate, etc., and particularly preferably methosulfate.

Preferred cosmetic agents are therefore characterized in that they contain a compound of formula (II)

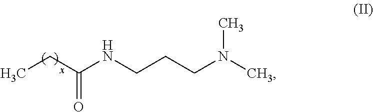

in which x represents 18, 19, 20, 21, 22, 23 or 24.

Compounds of the formula (II) where n=20 are particularly preferred. Highly preferred agents are characterized in that they always contain a compound of formula (I) together with a compound of the general formula (II).

Quaternary imidazoline compounds are a further group. The formula (Tkat4) shown below illustrates the structure of these compounds.

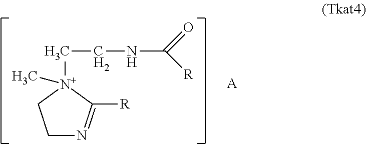

The R radicals, independently of one another, each stand for a saturated or unsaturated, linear or branched hydrocarbon group with a chain length of 8 to 30 carbon atoms. The preferred compounds of the formula (Tkat4) contain the same hydrocarbon radical for each instance of R. The chain length of the R radicals is preferably 12 to 21 carbon atoms. A represents an anion as described above. Particular examples according to the invention are available for example under the INCI names Quaternium-27, Quaternium-72, Quaternium-83 and Quaternium-91. Quaternium-91 is most preferred.

In a particularly preferred embodiment, the agent contains at least one amine and/or cationized amine, in particular an amidoamine and/or a cationized amidoamine having the following structural formula:

R1-NH—(CH₂)ₙ—N⁺R²R³R⁴A (Tkat5)
where R1 denotes an acyl or alkyl radical having 6 to 30 C atoms, which can be branched or unbranched, saturated or unsaturated, and wherein the acyl radical and/or alkyl radical can include at least one OH group, and

R2, R3 and R4 independently of one another are each
1) hydrogen or
2) an alkyl radical having 1 to 4 C atoms, which may be the same or different, saturated or unsaturated, and
3) a branched or unbranched hydroxyalkyl group having one to 4 carbon atoms with at least one and at most three hydroxyl groups, for example —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl radicals, and

A is an anion as described above and

n an integer between 1 and 10.

Preference is given to a cosmetic agent in which the amine and/or the quaternized amine according to the general formula (Tkat5) is an amidoamine and/or a quaternized amidoamine, in which R1 denotes a branched or unbranched, saturated or unsaturated acyl radical having 6 to 30 C atoms and may contain at least one OH group. Preference is given here to a fatty acid radical from oils and waxes, in particular from natural oils and waxes. Suitable examples include lanolin, beeswax, or candelilla wax.

Preference is also given to those amidoamines and/or quaternized amidoamines in which R2, R3 and/or R4 in the formula (Tkat5) denote a radical according to the general formula CH₂CH₂OR5, in which R5 can have the meaning of alkyl radicals having 1 to 4 carbon atoms, hydroxyethyl or hydrogen. The preferred value of n in the general formula (Tkat5) is a whole number between 2 and 5.

The alkylamidoamines are converted into a quaternary compound by protonation in a correspondingly acidic solution. Cationic alkyl amidoamines are preferred.

Examples of such commercial products are Witcamine® 100, Incromine® BB, Mackine® 401 and other Mackine® types, Adogen® S18V, and as permanent cationic aminoamines: Rewoquat® RTM 50, Empigen® CSC, Swanol® Lanoquat DES-50, Rewoquat® UTM 50, Schercoquat® BAS, Lexquat® AMG-BEO, or Incroquat® Behenyl H.

A further suitable fatty acid amide corresponds to the general formula (III):

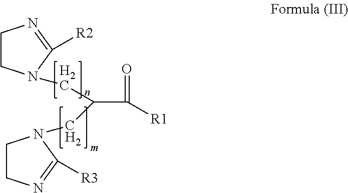

in which R1, R2 and R3 are independently a linear branched or unbranched C6 to C30, preferably C8 to C24, more preferably C12 to C22 and most preferably C12 to C18 alkyl or alkenyl group. R1 to R3 are preferably capryl, caprylyl, octyl, nonyl, decanyl, lauryl, myristyl, cetyl, stearyl, isostearyl, oleyl, behenyl, or arachidyl. Furthermore, R2 is particularly preferably the same as R3 and most preferably R1 is the same as R2, which is the same as R3. The letters n and m represent, independently of one another, integers from 1 to 10, preferably 2 to 6 and most preferably 2, 3 and/or 4, where the most preferably n=m. Most preferably, R1 is the same as R2, which is the same as R3 and selected from capryl, caprylyl, octyl, nonyl, decanyl, lauryl, myristyl, cetyl, stearyl, isostearyl, oleyl, behenyl, or arachidyl and n=m=2. Most preferably R1=R2=R3 and is selected from lauryl, myristyl, cetyl, stearyl, isostearyl, oleyl, behenyl or arachidyl, which is particularly preferably cetyl, stearyl, isostearyl, oleyl, or behenyl, and n=m=2. The most preferred compound of formula (I) is that which bears the INCI designation bis-ethyl (isostearoylimidazoline) isostearamides. The latter compound is commercially available under the trade name Keradyn® HH from Croda.

Further preferred quaternary ammonium compounds are cationic polymers.

The cationic polymers can be homopolymers or copolymers or polymers based on natural polymers, wherein the quaternary nitrogen groups are present either in the polymer chain or preferably as a substituent on one or more of the monomers. Suitable cationic monomers are unsaturated, radically polymerizable compounds which carry at least one cationic group, in particular ammonium-substituted vinyl monomers such as, for example, trialkyl methacryloxyalkylammonium, trialkylammonium and quaternary vinyl ammonium monomers with cyclic, cationic nitrogen-containing groups such as pyridinium, imidazolium or quaternary pyrrolidones, e.g., alkylvinylimidazolium, alkylvinylpyridinium, or alkylvinylpyrrolidone salts. The alkyl groups of these monomers are preferably lower alkyl groups, such as C1 to C7 alkyl groups, more preferably C1 to C3 alkyl groups.

The monomers containing ammonium groups can be copolymerized with non-cationic monomers. Suitable comonomers are, for example, acrylamide, methacrylamide; alkyl and dialkyl acrylamide, alkyl and dialkyl methacrylamide, alkyl acrylate, alkyl methacrylate, vinyl caprolactone, vinyl caprolactam, vinyl pyrrolidone, vinyl esters, e.g., vinyl acetate, vinyl alcohol, propylene glycol or ethylene glycol, where the alkyl groups of these monomers are preferably C1 to C7 alkyl groups, more preferably C1 to C3 alkyl groups.

Of these multiple polymers, the following ones have proven to be particularly suitable: homopolymers of the general formula —{CH₂—[CR¹COO—(CH₂)ₘN⁺R²R³R⁴]}ₙX⁻;
in which R¹=—H or —CH₃, R², R³ and R⁴, independently from one another, are selected from C1-4-alkyl, -alkenyl or -hydroxyalkyl groups, m=1, 2, 3 or 4, n is a natural number and
X⁻ is a physiologically acceptable organic or inorganic anion. In the context of these polymers, preference is given to those for which at least one of the following conditions applies: R¹ represents a methyl group, R², R³ and R⁴ represent methyl groups, or m has the value 2.

As a physiologically acceptable counter ion X—, for example, halide ions, sulfate ions, phosphate ions, methosulfate ions as well as organic ions such as lactate-, citrate-, tartrate- and acetate ions are considered. Methosulfate and halide ions are preferred, particularly chloride.

Suitable cationic polymers are, for example, copolymers according to the formula (Copo)

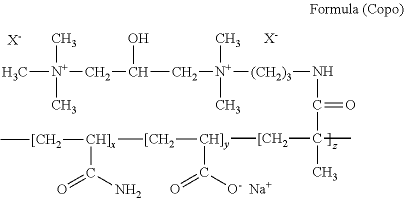

in which:

x+y+z=Q
Q represents values from 3 to 55,000, preferably from 10 to 25,000, particularly preferably from 50 to 15,000, more preferably from 100 to 10,000, even more preferably from 500 to 8000 and in particular from 1000 to 5000,
x represents (0 to 0.5) Q, preferably (0 to 0.3) Q and in particular the values 0, 1, 2, 3, 4, 5, where the value 0 is preferable,
y represents (0.1 to 0.95) Q, preferably (0.5 to 0.7) Q and in particular values from 1 to 24,000, preferably from 5 to 15,000, more preferably from 10 to 10,000 and in particular from 100 to 4800,
z represents (0.001 to 0.5) Q, preferably (0.1 to 0.5) Q, and in particular values from 1 to 12,500, preferably from 2 to 8000, more preferably from 3 to 4000 and especially from 5 to 2000.

Irrespective of which of the preferred copolymers of the formula (Copo) are used, preference is given to agents which are characterized in that the ratio of (y:z) is 4:1 to 1:2, preferably 4:1 to 1:1.

Irrespective of which copolymers are used in the agents, preference is given to hair treatment agents in which the copolymer has a molar mass from 10,000 to 20 million gmol-1, preferably from 100,000 to 10 million gmol-1, more preferably from 500,000 to 5 million gmol-1 and in particular from 1.1 million to 2.2 million gmol-1.

A highly preferred copolymer which is constructed as described above is commercially available under the designation polyquaternium-74.

A particularly suitable homopolymer is the poly(methacryloxyethyltrimethylammonium) chloride (in some cases crosslinked) having the INCI designation polyquaternium-37. Such products are commercially available, for example, under the designations Rheocare® CTH (Cosmetic Rheologies) and Synthalen® CR (3V Sigma).

The homopolymer is preferably used in the form of a non-aqueous polymer dispersion. Such polymer dispersions are commercially available under the designations Salcare® SC 95 and Salcare® SC 96.

Suitable cationic polymers derived from natural polymers are cationic derivatives of polysaccharides, for example cationic derivatives of cellulose, starch or guar. Chitosan and chitosan derivatives are also suitable. Cationic polysaccharides have the general formula:

G—O—B—N+RₐR_{b}R_{c}A⁻

G is an anhydroglucose radical, for example starch or cellulose anhydroglucose; B is a divalent linking group, for example alkylene, oxyalkylene, polyoxyalkylene or hydroxyalkylene;

Rₐ, R_{b} and R_{c} are, independently of one another, alkyl, aryl, alkylaryl, arylalkyl, alkoxyalkyl or alkoxyaryl, each having up to 18 C atoms, where the total number of carbon atoms in Rₐ, R_{b} and R_{c} is preferably at most 20;
A: is a conventional counter anion and is preferably chloride.

Cationic, i.e., quaternized celluloses are available on the market with different degrees of substitution, cationic charge density, nitrogen content and molecular weights. For example, polyquaternium-67 is offered commercially under the names SoftCat® Polymer SL or SoftCat® Polymer SK (Dow). A further highly preferred cellulose is offered under the trade name Mirustyle® CP from Croda. This is a trimonium and cocodimonium hydroxyethyl cellulose derivatized cellulose having the INCI designation polyquaternium 72. Polyquaternium-72 can be used both in solid form and already predissolved in aqueous solution.

Further cationic celluloses are Ucare® Polymer JR 400 (Dow, INCI designation polyquaternium-10) and Polymer Quatrisoft® LM-200 (Dow, INCI designation polyquaternium-24). Further commercial products are the compounds Celquat® H 100 and Celquat® L 200. Particularly preferred cationic celluloses are polyquaternium-24, polyquaternium-67 and polyquaternium-72.

Suitable cationic guar derivatives are marketed under the trade name Jaguar® and have the INCI designation guar hydroxypropyltrimonium chloride. Furthermore, particularly suitable cationic guar derivatives are also commercially available from Hercules under the name N-Hance®. Further cationic guar derivatives are marketed by BASF SE under the name Cosmedia®. A preferred cationic guar derivative is the commercial product AquaCat® from Hercules. This raw material is an already pre-dissolved cationic guar derivative. The cationic guar derivatives are preferred.

A suitable chitosan is marketed, for example, by Kyowa Oil& Fat, Japan under the trade name Flonac®. A preferred chitosan salt is chitosonium pyrrolidone carboxylate, which is marketed, for example, under the name Kytamer® PC by Amerchol, USA. Further chitosan derivatives are freely commercially available under the trade names Hydagen® CMF, Hydagen® HCMF and Chitolam® NB/101.

Another group of polymers that are of excellent use are polymers based on glucose. The following FIGURE shows such a cationic alkyl oligoglucoside.

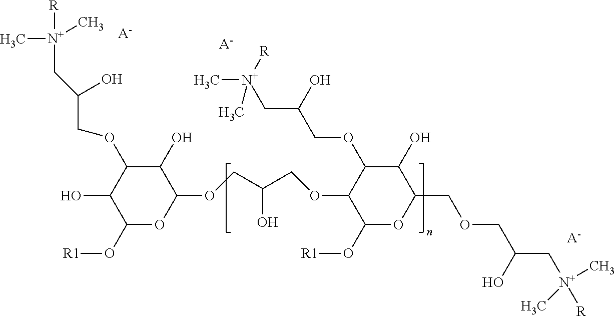

In the formula shown above, the radicals R, independently of one another, represent a linear or branched C6 to C30 alkyl radical, a linear or branched C6-C30 alkenyl radical; preferably the radical R represents a radical R selected from: lauryl, myristyl, cetyl, stearyl, oleyl, behenyl or arachidyl.

The radicals R1, independently of one another, represent a linear or branched C6 to C30 alkyl radical, a linear or branched C6 to C30 alkenyl radical; preferably the radical R represents a radical selected from: butyl, capryl, caprylyl, octyl, nonyl, decanyllauryl, myristyl, cetyl, stearyl, oleyl, behenyl or arachidyl. The radicals R1 are particularly preferably the same. More preferably, the radicals R1 are selected from technical mixtures of the fatty alcohol sections from C6/C8-fatty alcohols, C8/C10-fatty alcohols, C10/C12-fatty alcohols, C12/C14-fatty alcohols, C12/C18-fatty alcohols, and most preferable are those technical fatty alcohol sections which are of plant origin. The counterion to the cationic charge is a physiologically compatible anion, for example halide, methosulphate, phosphate, citrate, tartrate, etc. Preferably, the counterion is a halide, such as fluoride, chloride, bromide or methosulfate. The anion is most preferably chloride.

Particularly preferred examples of the cationic alkyl oligoglucosides are the compounds with the INCI designations polyquaternium-77, polyquaternium-78, polyquaternium-79, polyquaternium-80, polyquaternium-81, and polyquaternium-82. The cationic alkyl oligoglucosides with the designations polyquaternium-77, polyquaternium-81 and polyquaternium-82 are most preferred.

Such compounds can be purchased, for example, under the name Poly Suga® Quat from Colonial Chemical Inc.

A further preferred cationic polymer, comprises at least one structural unit of formula (IV), at least one structural unit of formula (V), at least one structural unit of formula (VI) and at least one structural unit of formula (VII),

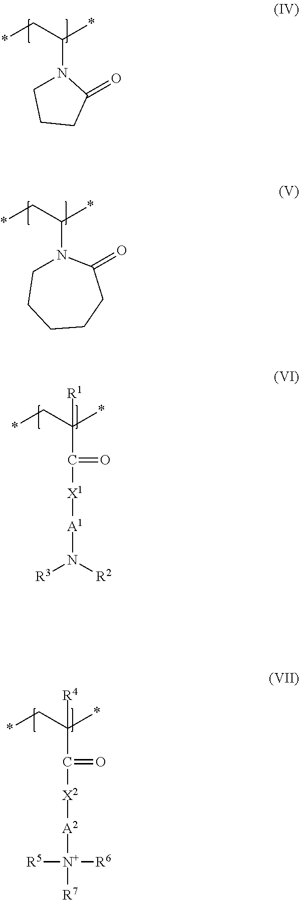

wherein
R¹ and R⁴ represent, independently of one another, a hydrogen atom or a methyl group,
X¹ and X² represent, independently of one another, an oxygen atom or an NH group,
A¹ and A² represent, independently of one another, an ethane-1,2-diyl group, a propane-1,3-diyl group or a butane-1,4-diyl group,
R², R³, R⁵ and R⁶ represent, independently of one another, a (C₁ to C₄) alkyl group,
R⁷ represents a (C₈ to C₃₀) alkyl group.

According to the above formulas and all of the following formulas, a chemical bond that is designated with the symbol * represents a free valance of the corresponding structure fragment.

To compensate for the positive polymer charge in the agent, all possible physiologically compatible anions are useful, such as chloride, bromide, hydrogen sulfate, methyl sulfate, ethyl sulfate, tetrafluoroborate, phosphate, hydrogen phosphate, dihydrogen phosphate or p-toluene sulfonate and triflate.

Examples of (C₁ to C₄) alkyl groups according to the invention are methyl, ethyl, propyl, isopropyl, butyl, sec-butyl, isobutyl and tert-butyl.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl) and docosyl (behenyl).

Subsequent cationic polymers can be used in the cosmetic agent if the cationic polymers, with respect to aforementioned formulae (IV) to (VII), perform one or more of the following features:
R¹ and R⁴ are each a methyl group,
X¹ represents an NH group,
X² represents an NH group,
A¹ and A², independently of one another, represent ethane-1,2-diyl or propane-1,3-diyl,
R², R³, R⁵ and R⁶, independently of one another, represent methyl or ethyl (particularly preferably methyl),
R⁷ represents a (C₁₀ to C₂₄) alkyl group, in particular for decyl (caprinyl), dodecyl (lauryl), tetradecyl (myristyl), hexadecyl (cetyl), octadecyl (stearyl), eicosyl (arachyl) or docosyl (behenyl).

It is preferable to select the structural unit of formula (VII) from at least one structural unit of formulae (VII-1) to (VII-8)

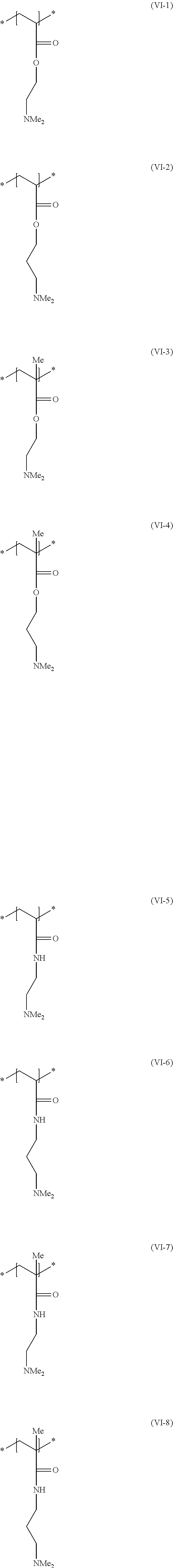

In addition, it proves particularly preferable to select the structural unit according to the formula (VI-7) and/or the formula (VI-8) as the structural unit of formula (VII). The structural unit of the formula (VI-8) is a very particularly preferred structural unit.

Further, it has been found to be preferable if the structural unit of the formula (VII) is selected from at least one structural unit of formulas (VII-1) to (VII-8)

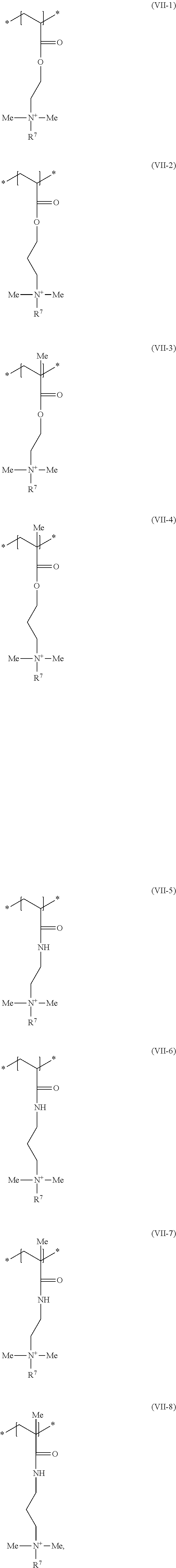

in which R⁷ in each case represents a (C₈ to C₃₀) alkyl group.

The structural units of the formula (VII-7) and/or the formula (VII-8) are considered, in turn, to be the particularly preferred structural unit of formula (VII), in which R7 represents, respectively octyl (capryl), decyl (caprinyl), dodecyl (lauryl), tetradecyl (myristyl), hexadecyl (cetyl), octadecyl (stearyl), eicosyl (arachyl) or docosyl (behenyl). The structural unit of formula (VII-8) represents a very particularly preferred structural unit of formula (VII).

A very particularly preferred cationic polymer contained in the agent comprises at least one structural unit of formula (IV), at least one structural unit of formula (V), at least one structural unit of formula (VI-8) and at least one structural unit of formula (VII-8),

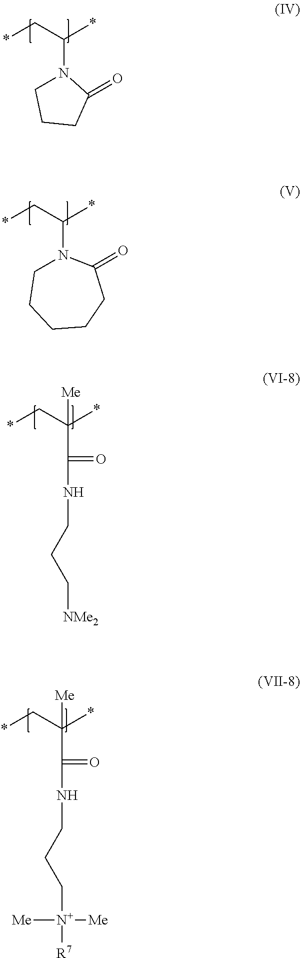

in which R⁷ represents octyl (capryl), decyl (caprinyl), dodecyl (lauryl), tetradecyl (myristyl), hexadecyl (cetyl), octadecyl (stearyl), eicosyl (arachyl) or docosyl (behenyl).

A very particularly preferred cationic polymer according to the invention is the copolymer of N-vinylpyrrolidone, N-vinylcaprolactam, N-(3-dimethylaminopropyl) methacrylamide and 3-(methacryloylamino) propyl-lauryl dimethylammonium chloride (INCI designation: polyquaternium-69), which is marketed, for example, under the trade name AquaStyle® 300 (28-32 wt. % active substance in an ethanol-water mixture, molecular weight 350,000) by the company ISP.

The polymers described so far represent only a portion of the usable polymers. In order not to describe all suitable cationic and/or amphoteric polymers in addition to their composition, the INCI-declarations of the preferred polymers are summarized. The preferred polymers bear the INCI designation:
polyquaternium-2, polyquaternium-4, polyquaternium-6, polyquaternium-7, polyquaternium-10, polyquaternium-11, polyquaternium-15, polyquaternium-16, polyquaternium-17, polyquaternium-18, polyquaternium-22, polyquaternium-24, polyquaternium-28, polyquaternium-32, polyquaternium-33, polyquaternium-34, polyquaternium-35, polyquaternium-37, polyquaternium-39, polyquaternium-41, polyquaternium-42, polyquaternium-44, polyquaternium-47, polyquaternium-55, polyquaternium-67, polyquaternium-68, polyquaternium-69, polyquaternium-72, polyquaternium-74, polyquaternium-76, polyquaternium-86, polyquaternium-89 and polyquaternium-95 as well as mixtures thereof.

Particularly preferred cationic polymers bear the INCI designations:
polyquaternium-2, polyquaternium-4, polyquaternium-11, polyquaternium-15, polyquaternium-16, polyquaternium-17, polyquaternium-18, polyquaternium-28, polyquaternium-32, polyquaternium-33, polyquaternium-34, polyquaternium-35, polyquaternium-39, polyquaternium-41, polyquaternium-42, polyquaternium-44, polyquaternium-47, polyquaternium-55, polyquaternium-68, polyquaternium-69, polyquaternium-74, polyquaternium-76, polyquaternium-86, polyquaternium-89 and polyquaternium-95 as well as mixtures thereof.

Most preferable are the cationic polymers with the designations:
polyquaternium-39, polyquaternium-44, polyquaternium-47, polyquaternium-55, polyquaternium-68, polyquaternium-69, polyquaternium-74, polyquaternium-76, polyquaternium-86, polyquaternium-89 and polyquaternium-95 as well as mixtures thereof.

The aforementioned cationic polymers can be used individually or in any combination with one another.

Further preferred cationic polymers are, for example,
cationized honey, for example the commercial product Honeyquat® 50,
polymeric dimethyldiallylammonium salts and copolymers thereof with esters and amides of acrylic acid and methacrylic acid. The products obtainable commercially under the designations Merquat®100 (poly(dimethyldiallylammonium chloride)) and Merquat®550 (dimethyldiallylammonium chloride/acrylamide copolymer) are examples of such cationic polymers with the INCI designation polyquaternium-7,
vinylpyrrolidone-vinylimidazolium methochloride copolymers, as are offered under the designations Luviquat® FC 370, FC 550 and the INCI designation Polyquaternium-16, as well as FC 905 and HM 552,
quaternized vinyl pyrrolidone/dimethylaminoethyl methacrylate, for example vinyl pyrrolidone/dimethylaminoethyl methacrylate methosulphate copolymer, which is marketed by Gaf Co., USA under the trade names Gafquat® 755 N and Gafquat® 734 and the INCI designation polyquaternium-11,
quaternized polyvinyl alcohol,
and the polymers known under the designations polyquaternium-2, polyquaternium-17, polyquaternium-18, and polyquaternium-27, having quaternary nitrogen atoms in the main polymer chain,
vinylpyrrolidone-vinylcaprolactam acrylate terpolymers, as are offered for example under the name Aquaflex® SF 40, having acrylic acid esters and acrylic acid amides as a third monomer unit.

amphoteric polymers are those polymers in which a cationic group derives from at least one of the following monomers:
(i) monomers having quaternary ammonium groups of the general formula (Mono1),

R¹—CH═CR²—CO—Z—(CₙH₂ₙ)—N⁽⁺⁾R²R³R⁴A⁽⁻⁾ (Mono1)
in which R¹ and R² represent, independently of one another, hydrogen or a methyl group, R³, R⁴ and R⁵ represent, independently of one another, alkyl groups having 1 to 4 carbon atoms, Z is an NH group or an oxygen atom, n is an integer from 2 to 5, and A⁽⁻⁾ is the anion of an organic or inorganic acid,
(ii) monomers having quaternary ammonium groups of the general formula (Mono2),

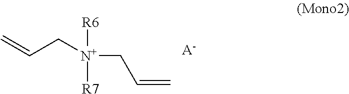

where R⁶ and R⁷, independently of one another represent a (C₁ to C₄) alkyl group, in particular a methyl group, and
A: is the anion of an organic or inorganic acid,
(iii) monomeric carboxylic acids of the general formula (Mono3),

R⁸—CH═CR⁹—COOH (Mono3)
in which R⁸ and R⁹ are, independently of one another, hydrogen or methyl groups.

Particular preference is given to those polymers in which monomers of the type (i) are used, in which R³, R⁴ and R⁵ are methyl groups, Z is an NH group and A⁽⁻⁾ is a halide, methoxysulfate or ethoxy sulfate Ion; acrylamidopropyl trimethyl ammonium chloride is a particularly preferred monomer (i). The monomer (ii) used for the aforementioned polymers mentioned is preferably acrylic acid.

Particularly preferred amphoteric polymers are copolymers made of at least one monomer (Mono1) or (Mono2) with the monomer (Mono3), in particular copolymers made of the monomers (Mono2) and (Mono3). Very particularly preferably used amphoteric polymers are copolymers made of diallyl dimethylammonium chloride and acrylic acid. These copolymers are marketed under the INCI designation polyquaternium-22, inter alia with the trade name Merquat® 280 (Nalco).

In addition, the amphoteric polymers, in addition to a monomer (Mono1) or (Mono2) and a monomer (Mono3), can additionally comprise a monomer (Mono4)
(iv) monomeric carboxylic acid amides of the general formula (Mono4),

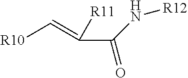

in which R¹⁰ and R¹¹ are, independently of one another, hydrogen or methyl groups, and R¹² represents a hydrogen atom or a (C₁- to C₈) alkyl group.

Very particularly preferably usable amphoteric polymers based on a comonomer (Mono4) are terpolymers of diallyldimethylammonium chloride, acrylamide and acrylic acid. These copolymers are marketed under the INCI designation polyquaternium-39, inter alia with the trade name Merquat® Plus 3330 (Nalco).

The amphoteric polymers can generally be used both directly and in salt form which is obtained by neutralization of the polymers, for example with an alkali hydroxide.

In a particularly preferred embodiment of the invention, the cosmetic agent contains a monoalkyl quat and/or a cationic guar derivative as quaternary compound.

A likewise preferred cosmetic agent for the nurturing and matting of keratinous fibers is characterized in that it comprises a silicone oil.

In the context of the present invention, the term “oil” is understood to mean a substance which is liquid at room temperature (25° C.). Furthermore, in the context of the invention, an oil has a solubility in water of less than 1 g/l, preferably less than 0.5 g/l, particularly preferably less than 0.1 g/l (measured at 25° C.).

The water solubility of the silicone oil can be determined, for example, by the following method: 1.0 g of the silicone oil is placed in a beaker. Then 1000 ml (1 liter) of water is added. A stirring bar is added, and the mixture is heated to 25° C. on a magnetic stirrer, while stirring. The mixture is stirred for 60 minutes. Thereafter, the aqueous mixture is visually assessed. If a second phase, i.e., in addition to the water phase, a separately present oil phase, is still visible after this period, then the solubility of the silicone oil is less than 1 g/l (1 gram/liter).

The silicone oils usable in cosmetic agents are polymeric compounds whose molecular weight is at least 500 g/mol, preferably at least 1000 g/mol, more preferably at least 2500 g/mol, and particularly preferably at least 5000 g/mol.

The silicone oils which can be used in the cosmetic agents comprise many Si—O repeat units, the Si atoms being able to bear organic radicals such as, for example, alkyl groups or substituted alkyl groups.

Corresponding to the high molecular weight of the silicone oils, these are based on more than 10 Si—O repeat units, preferably more than 50 Si—O repeat units and particularly preferably more than 100 Si—O repeat units.

It has proven particularly preferable to use in the cosmetic agent silicone oils having a viscosity from 5 to 3000 mm²/s, preferably 10 to 2000 mm²/s, further preferably from 10 to 1000 mm²/s, even more preferably from 10 to 500 mm²/s and very particularly preferably from 10 to 500 mm²/s (in each case measured according to ASTM standard D-445, 25° C.).

The ASTM standard D-445 is the standard method for measuring the kinematic viscosity of transparent and opaque fluids.

The viscosity was measured in particular according to ASTM standard D-446, version 06 (D445-06), published June 2006. In this measurement method, the time required for the defined volume of a liquid to flow under defined conditions through the capillary of a calibrated viscometer is measured. With regard to the details of the method, ASMT-D445, refer in particular to ASTM D445-06. Measurement temperature is 25° C. Suitable instruments (such as viscosimeters and thermometers and the corresponding calibrations) are specified in the method.

In principle, various silicone oils can be used in cosmetic agents, but the use of polydimethylsiloxanes has proven to be particularly advantageous with regard to the improvement of the feel of the hair and the reduction of its oily texture.

Suitable silicone oils from the group of linear polydimethylsiloxanes are compounds of the general structure (V)

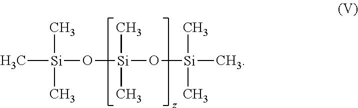

In this case, z is selected such that the dimethicones are liquid and preferably have the aforementioned very particularly well-suited viscosity ranges.

Preferably, z can be an integer from 50 to 100,000, more preferably from 100 to 50,000, particularly preferably from 500 to 50,000.

Corresponding dimethicones are commercially available from various manufacturers. Very particularly well suited is, for example, the dimethicone which is commercially available under the trade name Xiameters PMX 200 Silicone Fluid 50 CS from Dow Chemicals, the viscosity of which is 50 mm²/s (at 25° C.). This dimethicone is most preferred.

Another particularly well-suited dimethicone is Xiameter PMX 200 Silicone Fluid 100 CS, also available from Dow Corning, the viscosity of which is 100 mm²/s (measurement at 25° C.).

Another particularly well-suited dimethicone is Xiameter PMX 200 Silicone Fluid 350 CS, also available from Dow Corning, the viscosity of which is 350 mm²/s (at 25° C.).

Another particularly well suited dimethicone is Dow Corning 200 fluid 500 cSt available from Dow Corning, the viscosity of which is 500 mm²/s (at 25° C.).

The amount of nurturing ingredient—based on the total weight of the nurturing and matting cosmetic agent—is preferably 0.01 to 15 wt. %.

The amount of pigment suspension in the nurturing and matting cosmetic agent depends on the desired matting effect and can—based on the total weight thereof—be from 10 to 99 wt. %, preferably from 40 to 99 wt. %.

In addition to the at least one nurturing ingredient and the pigment suspension according to the invention, the nurturing and matting cosmetic agent can contain water and/or a mixture of water and at least one C₁-C₄ alcohol. Another advantageous ingredient of a nurturing and matting cosmetic agent is a fatty alcohol, in particular C₁₆-C₁₈ fatty alcohols.

The pigment suspension or the cosmetic agent can be used for matting keratinous fibers, in particular blond keratinous fibers, and very particularly preferably blond human hair.

Accordingly, a further subject matter of the application is the use of a pigment suspension according to the invention or of a cosmetic agent according to the invention for matting keratinous fibers.

### EXAMPLE

The following pigment suspension was produced:

| | Wt. % |
|---|---|
| Aluminum pigment suspension (active content: ~10 | 2 |
| wt. %, D₅₀ = 12 μm) | |
| PEG 400** | 87 |
| PEG 6000*** | 11 |

* INCI: Water (and) Isopropyl Alcohol (and) Aluminum Powder (CI 77000) (and) Isotridecyl Phosphate (and) Acetones (and) Isopropyl Acetate
**Polyethylene glycol having an average molecular weight of 400 g/mol
***Polyethylene glycol having an average molecular weight of 6,000 g/mol

The pigment suspension was massaged into a moist hair strands (Kerling, 10-0) and allowed to act for 1 minute. The pigment suspension was then rinsed with water.

After drying, the hair strands had a cool, matte ash-blond color.

1. A pigment suspension comprising:
at least one aluminum pigment; and
at least one polyethylene glycol having an average molecular weight of from 200 g/mol to 35,000 g/mol.

2. The pigment suspension of claim 1, wherein the at least one aluminum pigment has a substrate plate.

3. The pigment suspension of claim 1, wherein the at least one polyethylene glycol comprises at least two polyethylene glycols each having an average molecular weight of from 200 g/mol to 35,000 g/mol.

4. The pigment suspension of claim 3, wherein the at least one polyethylene glycol comprises at least one first polyethylene glycol having an average molecular weight of from 200 g/mol to 600 g/mol and at least one second polyethylene glycol having an average molecular weight of from 1000 g/mol to 35,000 g/mol.

5. The pigment suspension of claim 2, wherein the substrate plate comprises a vacuum metalized pigment.

6. The pigment suspension of claim 2, wherein the substrate plate comprises a lenticular substrate plate.

7. The pigment suspension of claim 2, wherein the substrate plate comprises a lamellar substrate plate.

8. A cosmetic agent, comprising
the pigment suspension of claim 1; and
a cleansing ingredient and/or nurturing ingredient.

9. The cosmetic agent of claim 8, wherein the cleansing ingredient comprises a cleansing surfactant selected from the group consisting of anionic surfactants, amphoteric/zwitterionic surfactants, and nonionic surfactants.

10. The cosmetic agent of claim 8, wherein the nurturing ingredient comprises at least one quaternary compound selected from the group consisting of:
i. monoalkylquats,
ii. esterquats,
iii. quaternary imidazolines of formula (Tkat4), 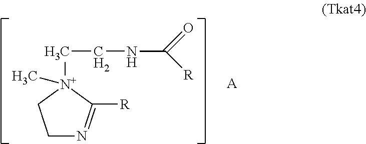
where the radicals R, independently of one another, each represent a saturated or unsaturated, linear or branched hydrocarbon radical having a chain length of 8 to 30 carbon atoms, and A represents a physiologically compatible anion,
iv. amines and/or cationized amines,
v. poly(methacryloyloxyethyl trimethylammonium compounds),
vi. quaternized cellulose derivatives,
vii. cationic alkylpolyglycosides,
viii. cationized honey,
ix. cationic guar derivatives,
x. chitosans,
xi. polymeric dimethyldiallylammonium salts and copolymers thereof with esters and amides of acrylic acid and methacrylic acid,
xii. copolymers of vinylpyrrolidone with quaternized derivatives of dialkylaminoalkyl acrylate and methacrylate,
xiii. vinylpyrrolidone-vinyl imidazolium methochloride copolymers,
xiv. quaternized polyvinyl alcohols,
xv. polyquaternium-74, and
xvi. mixtures thereof.

11. A method comprising matting keratinous fibers using the pigment suspension of claim 1.

12. The method of claim 11, wherein the keratinous fibers are blond human hairs.

13. A method comprising matting keratinous fibers using the cosmetic agent of claim 8.

14. The method of claim 13, wherein the keratinous fibers are blond human hairs.

15. The pigment suspension of claim 3, wherein the at least two polyethylene glycols are different from one another.

16. The pigment suspension of claim 4, wherein a weight ratio of the at least one second polyethylene glycol to the at least one first polyethylene glycol is from 15:1 to 1:1.

17. The cosmetic agent of claim 8, wherein the nurturing ingredient comprises at least one quaternary compound selected from the group consisting of polyquaternium-10, polyquaternium-24, polyquaternium-27, polyquaternium-67, polyquaternium-72, polyquaternium-7, and polyquaternium-11.

18. The pigment suspension of claim 2, wherein the substrate plate has an average diameter of from 2 μm to 200 μm.

19. The pigment suspension of claim 18, wherein the substrate plate has an average thickness of at most 150 nm.

20. The pigment suspension of claim 19, wherein a ratio of the average diameter to the average thickness is at least 80.
